(19) United States
(12) Patent Application Publication
(10) Pub. No.: US 2025/0235534 A1
(43) Pub. Date: Jul. 24, 2025

(54) An LTBR Agonist In Combination Therapy Against Cancer

(71) Applicant: VIB VZW, Gent (BE)

(72) Inventor: Pascal Merchiers, Kasterlee (BE)

(21) Appl. No.: 18/856,383

(22) PCT Filed: Apr. 13, 2023
(86) PCT No.: PCT/EP2023/059713

§ 371 Date: Oct. 11, 2024

(30) Foreign Application Priority Data
Apr. 13, 2022 (EP) 22168175.2

(51) Int. Cl. A61K 39/395; A61K 35/761; A61K 35/763

(52) U.S. Cl. CPC A61K 39/3955; A61K 35/761; A61K 35/763

(57) ABSTRACT

The present invention relates to a combination comprising an oncolytic virus and an LTBR agonist. The LTBR agonist may be administered separately or may be encoded by the oncolytic virus. Such a combination is particularly useful for use in the treatment of a cancer.

### CROSS-REFERENCE TO RELATED APPLICATIONS

This application is a national phase entry under 35 U.S.C. § 371 of International Patent Application PCT/EP2023/059713, filed Apr. 13, 2023, designating the United States of America and published in English as International Patent Publication WO 2023/198848 on Oct. 19, 2023, which claims the benefit under Article 8 of the Patent Cooperation Treaty to European Patent Application Serial No. 22168175.2, filed Apr. 13, 2022, the entireties of which are hereby incorporated by reference.

### FIELD OF THE INVENTION

The present invention relates to a combination comprising an oncolytic virus and an LTBR agonist. The LTBR agonist may be administered separately or may be encoded by the oncolytic virus. Such a combination is particularly useful for use in the treatment of a cancer.

### BACKGROUND OF THE INVENTION

Oncolytic viruses preferentially infect and kill cancer cells. In contrast to gene therapy where a virus is used as a mere carrier for transgene delivery, oncolytic virotherapy uses the virus itself as an active principle. They infect the tumor cell and then begin to replicate. The virus continues to replicate until it finally “lyses” (bursts) the host cell's membrane as the tumor cell can no longer contain the virus. As the infected dividing cells are destroyed by lysis, they release new infectious virus particles to infect the surrounding dividing cells. Cancer cells are ideal hosts for many viruses because they have viral infection protection mechanisms, such as the antiviral interferon pathway, inactivated or have mutated tumor suppressor genes that enable viral replication to proceed unhindered. A number of viruses including adenovirus, reovirus, measles, herpes simplex, Newcastle disease virus, and vaccinia have been clinically tested as oncolytic agents. Most current oncolytic viruses are engineered for tumor selectivity, although there are naturally occurring examples such as reovirus and the senecavirus. Modifications towards tumor selectivity include functional deletions in essential viral genes, the use of tumor-or tissue-specific promoters to control the viral gene expression and viral coat protein engineering to redirect virus to the cancer cell surface.

To date, two genetically engineered oncolytic viruses have been approved. One is H101 (Oncorine), which was approved in China for head and neck cancer and esophagus cancer. The other is talimogene laherparepvec (T-Vec, IMLYGIC), which was approved for treatment of inoperably melanoma. H101 virus is an E1B-deleted adenovirus, which allows it to replicate in p53 deficient cancer cells. Talimogene laherparepvec is a genetically engineered herpes simplex virus. The virus invades both cancerous and healthy cells, but it cannot productively replicate in healthy tissue because it lacks Infected cell protein 34.5 (ICP34.5). ICP34.5 blocks the cellular stress response against viral infection, allowing the virus to hijack the cell's translation machinery to replicate itself. As talimogene laherparepvec lacks funtional ICP34.5, it cannot replicate in normal tissue. However, in many cancer cells the stress response is already disrupted, so it can replicate in tumors.

As in several oncolytic viruses that are undergoing clinical development, talimogene laherparepvec has furthermore been engineered to induce an increased immune response. Infected cell protein 47 (ICP47) was deleted from talimogene laherparepvec because it is a protein encoded by the Herpes simplex virus that allows it to evade the human immune system's CD8 T-cell response by interfering with an infected cell's ability to show viral epitopes to T cells. Additionally, talimogene laherparepvec carries the transgene for granulocyte-macrophage colony-stimulating factor (GM-CSF), such that viral infected cells produce the immune stimulatory protein human GM-CSF. GM-CSF is secreted or released when the cancer cell bursts, attracting dendritic cells to the site, which pick up antigens, process them, and then present them on their surface to cytotoxic (killer) T cells which in turn sets off an immune response. As cancer cells are lysed, cancer antigens are released and result in induction of a systemic anti-tumor immune response and an effector T-cell response. Through this mechanism, local intratumoral injections with talimogene laherparepvec can act on remote lesions via induction of systemic antitumor immunity and thereby prolong survival. Mice that had complete regression of their primary tumors following treatment were found to be resistant to subsequent tumor re-challenge.

Despite the promise of oncolytic viral therapy, it appears that its full potential hasn't been reached yet and that therapeutic efficacy is often limited. The two main approaches that are currently investigated to increase oncolytic virotherapy efficacy are the combination of the virus with anti-angiogenic therapeutics or immune checkpoint inhibitors. These then either administered separately, e.g. systemically, or included as transgenes in the oncolytic virus. Despite these efforts, further efficacy improvements are required. It would be particularly beneficial to increase the systemic immune response, such that e.g. remote metastases are attacked efficiently for a sufficient period of time.

### SUMMARY OF THE INVENTION

The inventors have now surprisingly found that combining an oncolytic virus with a Lymphtoxin Beta Receptor (LTBR) agonist fulfils the above-mentioned needs. In particular, the inventors have surprisingly found that a synergistic effect is observed when the oncolytic virotherapy and the LTBR agonist as defined in the combination of the present invention are used. More in particular, the inventors have found that the combination increases the patient anti-cancer immune response compared to oncolytic virus monotherapy. The combination of the present invention therefore provides an improved tumour therapy.

Therefore, it is an object of the invention to provide an LTBR agonist for use in the treatment of cancer, wherein the treatment comprises administration of an oncolytic virus. The LTBR agonist may be administered as a molecule that has LTBR agonistic activity by itself or through administration of a nucleic acid encoding the LTBR agonist. Upon administration of the nucleic acid, in vivo expression may produce the LTBR agonist. Therefore, the present invention provides an LTBR agonist or a nucleic acid encoding an LTBR agonist for use in the treatment of a cancer, wherein the treatment comprises administration of an oncolytic virus. The LTBR agonist or the nucleic acid encoding an LTRB agonist may be comprised by the oncolytic virus. For example, the LTBR agonist may be part of or attached to the viral capsid. In case a nucleic acid encoding the LTBR agonist is used, the nucleic acid can be administered separately or can be added as a transgene in the oncolytic virus. Therefore, in a particular embodiment, the nucleic acid encoding the LTBR agonist is comprised by the oncolytic virus.

In another embodiment, the LTBR agonist or the nucleic acid encoding an LTBR agonist is separate from the oncolytic virus. In such embodiment, the LTBR agonist or the nucleic acid encoding the LTBR agonist may be administered separately from or simultaneously with the oncolytic virus.

In a preferred embodiment, the LTBR agonist of the invention comprises a proteinaceous moiety that binds to LTBR. In a further embodiment, the LTBR agonist comprises an antibody or fragment thereof that binds to LTBR, such as a single domain antibody moiety that binds to LTBR.

As can be derived from the above, the present invention thus also provides an oncolytic virus comprising a nucleic acid sequence encoding an LTBR agonist. The oncolytic virus will typically be designed such that the LTBR agonist is expressed in cells, particularly cancer cells, that have been infected with the oncolytic virus. In a further embodiment, the oncolytic virus comprises a nucleic acid sequence that encodes an LTBR agonist that comprises a single domain antibody moiety that binds to LTBR.

In principle, any oncolytic virus can be used for the present invention. In a particular embodiment, the oncolytic virus is a modified herpes simplex virus or adenovirus.

In another embodiment, the present invention provides a combination of (a) an LTBR agonist or a nucleic acid encoding an LTBR agonist, and (b) an oncolytic virus.

Another object of the invention is to provide a composition comprising the combination of the present invention. In a further embodiment, the present invention provides a pharmaceutical composition comprising the oncolytic virus as defined herein or the combination as defined herein. Such pharmaceutical composition may further comprise one or more pharmaceutically acceptable excipients. The present invention further provides a pharmaceutical composition as defined herein for use as a medicine, in particular for use in the treatment of cancer.

Preferably, the cancer is selected from the group consisting of breast cancer, uterine corpus cancer, lung cancer, stomach cancer, head and neck squamous cell carcinoma, skin cancer, colorectal cancer, and kidney cancer. More preferably, the cancer is skin cancer, particularly melanoma, more particularly melanoma that is metastatic. In another preferred embodiment, the cancer is head and neck or oesophageal cancer. In a further embodiment, the cancer is head and neck squamous cell carcinoma.

Yet another object of the present invention is to provide a combination therapy, comprising administration of an LTBR agonist or a nucleic acid encoding an LTBR agonist, and an oncolytic virus. As described herein, the LTBR agonist or the nucleic acid encoding an LTBR agonist may be comprised by the oncolytic virus.

In a particular embodiment, the LTBR agonist is an LTBR agonistic antibody.

A further object of the present invention is an oncolytic virus for use in the treatment of a cancer, wherein the treatment further comprises the administration of an LTBR agonist.

In addition to the oncolytic virus and the LTBR agonist, the therapy may comprise a further active ingredient. In a further embodiment, the further active ingredient is a checkpoint inhibitor. A checkpoint inhibitor is a compound that blocks checkpoint proteins from binding to their partner proteins thereby activating the immune system function. Preferably the checkpoint inhibitor blocks proteins selected from the group consisting of PD-1, PD-L1, CTLA4, B7-1 and B7-2. More preferably the checkpoint inhibitor blocks PD-1 or PD-L1. Preferred examples include anti-PD-1 and anti-PD-L1 antibodies. Preferred immune checkpoint inhibitors for use in the present invention are selected from nivolumab, pembrolizumab, atezolizumab, avelumab, durvalumab, cemiplimab, JTX-4014, spartalizumab, camrelizumab, sintilimab, tislelizumab, toripalimab, dostarlimab, INCMGA00012, AMP-224, AMP-514, KN035, AUNP12, CK-301, CA-170, and BMS-986189.

Alternatively, the further active ingredient is an anti-angiogenic agent, such as a VEGF inhibitor.

In yet another embodiment, the treatment is combined with another anti-cancer therapy. For example, the treatment according to the invention may particularly combined with radiotherapy or chemotherapy.

### DETAILED DESCRIPTION OF THE INVENTION

The present invention will be described in the following with respect to particular embodiments and with reference to certain drawings but the invention is not limited thereto.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclature used in connection with, and techniques of, molecular biology, immunology, microbiology, genetics and protein and nucleic acid chemistry described herein are those well-known and commonly used in the art.

As described herein before, the present invention provides a combination comprising an oncolytic virus and an LTBR agonist. Such a combination is particularly useful due to the synergistic effect observed when the oncolytic virus and the LTBR agonist as defined in the combination of the present invention are administrated as a combined cancer therapy.

### LTBR Agonist

As described herein, the term “LTBR agonist” refers to ligands specific for the receptor LTBR, which are compounds having the action of binding to the receptor, thus specifically stimulating ligand-dependent receptor activity (as differentiated from the baseline level determined in the absence of any ligand) or nucleic acid molecules encoding such ligands. This action is also simply referred to as a receptor-stimulating action or a receptor-activating action. Moreover, as synonyms for “agonist”, “activator”, “stimulator”, “receptor-activating ligand. Agonists include natural compounds, semisynthetic compounds derived from natural compounds, and synthetic compounds. LTBR agonists are known in the field and they are involved in the induction of high endothelial vesicles (HEVs) and tertiary lymphocyte structures (TLSs).

Within the context of the present invention, LTBR agonist therefore also comprises nucleic acids encoding an LTBR ligand that agonizes LTBR. Whereas the description will often refer to an LTBR agonist or a nucleic acid encoding an LTBR agonist, the mentioning of LTBR agonist without specification of a nucleic acid encoding an LTBR agonist comprises both option, unless the context clearly dictates otherwise. That is to say, LTBR agonist as used herein refers to both direct and indirect LTBR agonists. The term “direct LTBR agonist” as used herein refers to a ligand specific for the receptor LTBR. The term “indirect LTBR agonist” as used herein refers to nucleic acids that encode a direct LTBR agonist.

Within the context of the present invention, the terms “nucleic acid”, “nucleic acid molecule”, “polynucleotide” and “nucleotide sequence” are used interchangeably and define a polymer of any length of either polydeoxyribonucleotides (DNA) (e.g. cDNA, genomic DNA, plasmids, vectors, viral genomes, isolated DNA, probes, primers and any mixture thereof) or polyribonucleotides (RNA) (e.g. mRNA, antisense RNA, SIRNA) or mixed polyribo-polydeoxyribonucleotides. They encompass single or double-stranded, linear or circular, natural or synthetic, modified or unmodified polynucleotides. Moreover, a polynucleotide may comprise non-naturally occurring nucleotides and may be interrupted by non-nucleotide components.

In a particular embodiment, the LTBR agonist of the invention is administered as a therapeutic nucleic acid. The term “therapeutic nucleic acid” used herein refers to any nucleic acid molecule that has a therapeutic effect when introduced into a eukaryotic organism (e.g., a mammal such as human) and includes DNA and RNA molecules encoding the LTBR agonist of the invention. As is known to the skilled person, the nucleic acid may comprise elements that induce transcription and/or translation of the nucleic acid or that increases ex and/or in vivo stability of the nucleic acid.

LTBR, also known as tumor necrosis factor receptor superfamily member 3 (TNFRSF3), is a cell surface receptor for lymphotoxin involved in apoptosis and cytokine release. It is a member of the tumor necrosis factor receptor superfamily. It is expressed on the surface of most cell types, including cells of epithelial and myeloid lineages, but not on T and B lymphocytes. The protein specifically binds the lymphotoxin membrane form (a complex of lymphotoxin-alpha and lymphtoxin-beta). The encoded protein and its ligand play a role in the development and organization of lymphoid tissue.

Lymphotoxin-alpha/beta/beta (Lymphotoxin-αββ) is a heterotrimeric species comprised of one subunit or copy of lymphotoxin-alpha and two subunits or copies of lymphotoxin-beta. Lymphotoxin-αββ binds to the lymphotoxin-beta receptor (LTBR). The activation of LTBR initiates a signaling event resulting in the expression of chemokines, including but not limited to, CXCL12, CXCL13, CCL19, and CCL21. These chemokines serve to induce the migration of dendritic cells, T-cells, and B-cells to establish the germinal center. Lymphotoxin-αββ is thus an LTBR agonist and HEV inducer suitable for application in the present invention.

LIGHT, also known as tumor necrosis factor superfamily member 14 (TNFSF14), is a member of the TNF superfamily, and its receptors have been identified as lymphotoxin beta receptor (LTBR), herpes virus entry mediator (HVEM), and decoy receptor 3 (DcR3). LIGHT stands for “homologous to lymphotoxin, exhibits inducible expression and competes with HSV glycoprotein D for binding to herpesvirus entry mediator, a receptor expressed on T lymphocytes”. In the cluster of differentiation terminology it is classified as CD258. This protein may function as a costimulatory factor for the activation of lymphoid cells. It is a known LTBR agonist and HEV inducer.

As will be understood by the skilled person, in principle any type of agonist of LTBR can be used in the present invention and different types of agonists are readily available to the skilled person or can be generated using the typical knowledge in the art, including small molecules and biologics or biologic-derived molecules. In a particular embodiment, the binding moiety of the LTBR agonist is proteinaceous, more particularly an LTBR agonistic polypeptide. In a further embodiment, the binding moiety of the LTBR agonist is antibody based or non-antibody based, preferably antibody based. Non-antibody based agonists include, but are not limited to, affibodies, Kunitz domain peptides, monobodies (adnectins), anticalins, designed ankyrin repeat domains (DARPins), centyrins, fynomers, avimers; affilins; affitins, peptides and the like.

In a particular embodiment, the LTBR agonist is selected from Lymphotoxin-αββ, LIGHT, or LTBR binding fragments or mimetics thereof. In another embodiment, the LTBR agonist comprises lymphotoxin alpha or lymphotoxin beta. In a further embodiment, the LTBR agonist is a fusion peptide comprising lymphotoxin alpha and lymphotoxin beta, in particular one lymphotoxin alpha part and two lymphotoxin beta parts. Such LTBR agonists are, for example, disclosed in WO2018119118 A1 and WO9622788 A1, which are incorporated herein by reference. In a particular embodiment, the LTBR agonist comprises SEQ ID NO: 16, SEQ ID NO: 17, or SEQ ID NO: 18 of WO2018119118 A1.

LIGHT and LIGHT mimetic peptides are also known in the art, e.g. from WO2018119118 A1. In certain embodiments, the LTBR agonist comprises LIGHT (e.g., human LIGHT) or a fragment thereof. As a non-limiting example, the LTBR-binding moiety may comprise the extracellular domain of LIGHT or a fragment thereof. In certain embodiments, the LTBR agonist comprises a LIGHT homotrimer (e.g., a single-chain LIGHT homotrimer). For instance, the LTBR agonist may comprise the extracellular domain of human LIGHT, a variant thereof having at least 80% sequence identity to the extracellular domain of human LIGHT, or a fragment thereof. In certain embodiments, the LTBR agonist may comprise a polypeptide (e.g., a LIGHT homotrimer) having at least about 80%, at least about 90%, at least about 95%, at least about 98%, or 100% sequence identity to SEQ ID NO:85 of WO2018119118 A1. In some embodiments, the LTBR agonist is a single-chain polypeptide. In certain embodiments, the LTBR agonist comprises a polypeptide having at least about 90%, at least about 95%, or at least about 98% sequence identity to SEQ ID NO:86 of WO2018119118 A1. For example, the LTBR agonist may comprise SEQ ID NO: 86 of WO2018119118 A1. In some embodiments, the LTBR agonist comprises a mutant LIGHT homotrimer that has reduced the ability to bind to or activate HVEM.

In a particular embodiment, the LTBR agonist specifically binds to LTBR, preferably human LTBR. “Specific binding”, “bind specifically”, and “specifically bind” is particularly understood to mean that the LTBR agonist has a dissociation constant (K_{d}) for LTBR of less than about 10⁻⁶ M, 10⁻⁷M, 10⁻⁸ M, 10⁻⁹ M, 10⁻¹⁰ M, 10⁻¹¹ M, 10⁻¹² M or 10⁻¹³ M. In a preferred embodiment, the dissociation constant is less than 10⁻⁸ M, for instance in the range of 10⁻⁹ M, 10⁻¹⁰ M, 10⁻¹¹ M, 10⁻¹² M or 10⁻¹³ M. LTBR agonist affinities towards LTBR may be determined by a surface plasmon resonance based assay (such as the BIAcore assay as described in PCT Application Publication No. WO2005/012359) using viral like particles; cellular enzyme-linked immunoabsorbent assay (ELISA); and fluorescent activated cell sorting (FACS) read outs for example. A preferred method for determining apparent Kd or EC50 values is by using FACS at 21° C. with cells overexpressing LTBR, in particular human LTBR.

In a preferred embodiment, the agonist comprises an LTBR agonistic moiety that is an antibody or active antibody fragment. In a further aspect of the invention, the agonist is an antibody (“agonistic antibody”). Agonistic antibodies that specifically bind LTBR are known in the art. For example, see WO2006/114284 A2, WO2004/058191 A2, and WO02/30986 A2, each of which is hereby incorporated by reference herein. In a further aspect of the invention the antibody is monoclonal. The antibody may additionally or alternatively be humanised or human. In a further aspect, the antibody is human, or in any case an antibody that has a format and features allowing its use and administration in human subjects. Antibodies may be derived from any species, including but not limited to mouse, rat, chicken, rabbit, goat, bovine, non-human primate, human, dromedary, camel, llama, alpaca, and shark.

In one aspect of the invention, the LTBR agonist comprises an active antibody fragment.

In another embodiment, the LTBR agonist as detailed above, comprises at least one single domain antibody moiety. Preferably, the LTBR agonist comprises at least two single domain antibody moieties.

In a further embodiment of the present invention, the LTBR agonist, as detailed above, comprises at least one Fc region moiety and at least two single domain antibody moieties that bind to LTBR. Preferably, the LTBR agonist is a genetically engineered polypeptide that comprises at least one Fc region moiety and at least two single domain antibody moieties that bind to LTBR, joined together by a peptide linker. The amino acid sequence of the Fc region moiety and/or the single domain antibody moiety region(s) may be humanized to reduce immunogenicity for humans.

In particular, the single domain antibody may be a Nanobody® (as defined herein) or a suitable fragment thereof (Note: Nanobody®, Nanobodies® and Nanoclone® are registered trademarks of Ablynx N.V., a Sanofi Company). Furthermore, as for full-size antibodies, single variable domains such as VHHs and Nanobodies® can be subjected to humanization and give humanized single domain antibodies. In another particular embodiment, the LTBR agonist does not comprise an Fc domain. In a further particular embodiment, the LTBR agonist comprises one or more single domain antibody moieties and does not comprise an Fc domain. Techniques for generating LTBR agonists are available to the person skilled in the art.

As described herein, the terms “antibody”, “antibody fragment” and “active antibody fragment” refer to a protein comprising an immunoglobulin (Ig) domain or an antigen-binding domain capable of specifically binding the antigen, in particular LTBR. “Antibodies” can further be intact immunoglobulins derived from natural sources or from recombinant sources and can be immunoreactive portions of intact immunoglobulins. Antibodies may be multimers, such as tetramers, of immunoglobulin molecules. In a further aspect of the invention, the LTBR agonist is an antibody. In a further aspect of the invention the antibody is monoclonal. The antibody may additionally or alternatively be humanised or human. In a further aspect, the antibody is human, or in any case an antibody that has a format and features allowing its use and administration in human subjects. Antibodies may be derived from any species, including but not limited to mouse, rat, chicken, rabbit, goat, bovine, non-human primate, human, dromedary, camel, llama, alpaca, and shark.

The term “antigen-binding fragment” is intended to refer to an antigen-binding portion of said intact polyclonal or monoclonal antibodies that retains the ability to specifically bind to a target antigen or a single chain thereof, fusion proteins comprising an antibody, and any other modified configuration of the immunoglobulin molecule that comprises an antigen recognition site. The antigen-binding fragment comprises, but not limited to Fab; Fab′; F(ab′)₂; a Fc fragment; a single domain antibody (sdAb or dAb) fragment. These fragments are derived from intact antibodies by using conventional methods in the art, for example by proteolytic cleavage with enzymes such as papain (to produce Fab fragments) or pepsin (to produce F(ab′)₂ fragments). As used herein, antigen-binding fragment also refers to fusion proteins comprising heavy and/or light chain variable regions, such as single-chain variable fragments (scFv).

As used herein, the term “monoclonal antibody” refers to an antibody composition having a homogeneous antibody population. It is understood that monoclonal antibodies are highly specific, being directed against a single antigenic site. Furthermore, in contrast to conventional antibody (polyclonal) preparations which typically include different antibodies directed against different determinants (epitopes), each monoclonal antibody is directed against a single determinant on the antigen. The LTBR agonists of the invention preferably comprise a monoclonal antibody moiety that binds to LTBR, preferably human LTBR.

As used herein, the term “humanized antibody” refers to an antibody produced by molecular modelling techniques to identify an optimal combination of human and non-human (such as mouse or rabbits) antibody sequences, that is, a combination in which the human content of the antibody is maximized while causing little or no loss of the binding affinity attributable to the variable region of the non-human antibody. For example, a humanized antibody, also known as a chimeric antibody comprises the amino acid sequence of a human framework region and of a constant region from a human antibody to “humanize” or render non-immunogenic the complementarity determining regions (CDRs) from a non-human antibody.

As used herein, the term “human antibody” means an antibody having an amino acid sequence corresponding to that of an antibody that can be produced by a human and/or which has been made using any of the techniques for making human antibodies known to a skilled person in the art or disclosed herein. It is also understood that the term “human antibody” encompasses antibodies comprising at least one human heavy chain polypeptide or at least one human light chain polypeptide. One such example is an antibody comprising murine light chain and human heavy chain polypeptides.

In one aspect of the invention, the LTBR agonist comprises an active antibody fragment. The term “active antibody fragment” refers to a portion of any antibody or antibody-like structure that by itself has high affinity for an antigenic determinant, or epitope, and contains one or more antigen-binding sites, e.g. complementary-determining-regions (CDRs), accounting for such specificity. Non-limiting examples include immunoglobulin domains, Fab, F(ab)′₂, scFv, heavy-light chain dimers, immunoglobulin single variable domains, single domain antibodies (sdAb or dAb), Nanobodies®, and single chain structures, such as complete light chain or complete heavy chain, as well as antibody constant domains that have been engineered to bind to an antigen. An additional requirement for the “activity” of said fragments in the light of the present invention is that said fragments are capable of agonizing LTBR. The term “immunoglobulin (Ig) domain” or more specifically “immunoglobulin variable domain” (abbreviated as “IVD”) means an immunoglobulin domain essentially consisting of framework regions interrupted by complementary determining regions. Typically, immunoglobulin domains consist essentially of four “framework regions” which are referred in the art and below as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred in the art and herein below as “complementarity determining region 1” or “CDR1”; as “complementarity determining region 2” or “CDR2”; and as “complementarity determining region 3” or “CDR3”, respectively. Thus the general structure or sequence of an immunoglobulin variable domain can be indicated as follows: FR1-CDR1-FR2-CDR2-FR3-CDR3-FR4. It is the immunoglobulin variable domain(s) (IVDs) that confer specificity to an antibody for the antigen by carrying the antigen-binding site. Typically, in conventional immunoglobulins, an heavy chain variable domain (VH) and a light chain variable domain (VL) interact to form an antigen binding site. In this case the complementary determining regions (CDRs) of both VH and VL will contribute to the antigen binding site, i.e. a total of 6 CDRs will be involved in antigen binding site formation. In view of the above definition, the antigen-binding domain of a conventional 4-chain antibody (such as IgG, IgM, IgA, IgD or IgE molecule; known in the art) or of a Fab fragment, a F(ab′)₂ fragment, an Fv fragment such as a disulphide linked Fv or scFv fragment, or a diabody (all known in the art) derived from such conventional 4-chain antibody, with binding to the respective epitope of an antigen by a pair of (associated) immunoglobulin domains such as light and heavy chain variable domains, i.e., by a VH-VL pair of immunoglobulin domains, which jointly bind to an epitope of the respective antigen. A single domain antibody (sdAb) as used herein, refers to a protein with an amino acid sequence comprising 4 framework regions (FR) and 3 complementarity determining regions (CDRs) according to the format FR1-CDR1-FR2-CDR2-FR3-CDR3-FR4. Single domain antibodies of this invention are equivalent to “immunoglobulin single variable domains” (abbreviated as “ISVD”) and refers to molecules wherein the antigen binding site is present on, and formed by, a single immunoglobulin domain. This sets single domain antibodies apart from “conventional” antibodies or their fragments, wherein two immunoglobulin domains, in particular two variable domains interact to form an antigen binding site. The binding site of a single domain antibody is formed by a single VH/VHH or VL domain. Hence, the antigen binding site of a single domain antibody is formed by no more than 3 CDRs. As such a single domain may be a light chain variable domain sequence. (e.g. a VL-sequence) or a suitable fragment thereof; or a heavy chain variable domain sequence (e.g. a VH-sequence or VHH sequence) or a suitable fragment thereof; as long as it is capable of forming a single antigen binding unit (i.e., a functional antigen binding unit that essentially consists of a single variable domain, such that the single antigen binding domain does not need to interact with another variable domain to form a functional antigen binding unit).

In particular, the single domain antibody may be a Nanobody® (as defined herein) or a suitable fragment thereof (Note: Nanobody®, Nanobodies® and Nanoclone® are registered trademarks of Ablynx N.V., a Sanofi Company). For general description of Nanobodies® reference is made to the further description below, and described in the prior art such as e.g. WO2008/020079. “VHH domains”, also known as VHHs, VHH antibody fragments and VHH antibodies, have originally been described as the antigen binding immunoglobulin (Ig) (variable) domain of “heavy chain antibodies” (i.e. of “antibodies devoid of light chains”; see e.g. Hamers-Casterman et al., Nature 363:446-8 (1993)). The term “VHH domain” has been chosen to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHHs and Nanobodies®, reference is made to the review article by Muyldermans (Reviews in Molecular Biotechnology 74:277-302, 2001), as well as to the following patent applications, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and Ablynx N.V.; WO 01/90190 by the National Research Council of Canada; WO 03/025020 (=EP 1433793) by the Institute of Antibodies; as well as WO 04/041867, WO 04/041862, WO 04/041865, WO 04/041863, WO 04/062551, WO 05/044858, WO 06/40153, WO 06/079372, WO 06/122786, WO 06/122787 and WO 06/122825, by Ablynx N.V. and the further published patent applications by Ablynx N.V. As described in these references, Nanobody® (in particular VHH sequences and partially humanized Nanobody*) can in particular be characterized by the presence of one or more “Hallmark residues” in one or more of the framework sequences. A further description of the Nanobody “, including humanization and/or camelization of Nanobody, as well as other modifications, parts or fragments, derivatives or “Nanobody fusions”, multivalent or multispecific constructs (including some non-limiting examples of linker sequences) and different modifications to increase the half-life of the Nanobody” and their preparations can be found e.g. in WO 08/101985 and WO 08/142164. VHHs and Nanobodies are among the smallest antigen binding fragment that completely retains the binding affinity and specificity of a full-length antibody (see e.g. Greenberg et al., Nature 374:168-73 (1995); Hassanzadeh-Ghassabeh et al., Nanomedicine (Lond), 8:1013-26 (2013)).

Furthermore, as for full-size antibodies, single variable domains such as VHHs and Nanobodies® can be subjected to humanization, i.e. increase the degree of sequence identity with the closest human germline sequence. In particular, humanized immunoglobulin single variable domains, such as VHHs and Nanobodies® may be single domain antibodies in which at least one single amino acid residue is present (and in particular, at least one framework residue) that is and/or that corresponds to a humanizing substitution (as defined further herein). Potentially useful humanizing substitutions can be ascertained by comparing the sequence of the framework regions of a naturally occurring VHH sequence with the corresponding framework sequence of one or more closely related human VH sequences, after which one or more of the potentially useful humanizing substitutions (or combinations thereof) thus determined can be introduced into said VHH sequence and the resulting humanized VHH sequences can be tested for affinity for the target, for stability, for ease and level of expression, and/or for other desired properties. In this way, by means of a limited degree of trial and error, other suitable humanizing substitutions (or suitable combinations thereof) can be determined by the skilled person.

Humanized single domain antibodies, in particular VHHs and Nanobodies®, may have several advantages, such as a reduced immunogenicity, compared to the corresponding naturally occurring VHH domains. By humanized is meant mutated so that immunogenicity upon administration in human patients is minor or non-existent. The humanizing substitutions should be chosen such that the resulting humanized amino acid sequence and/or VHH still retains the favourable properties of the VHH, such as the antigen-binding capacity. Based on the description provided herein, the skilled person will be able to select humanizing substitutions or suitable combinations of humanizing substitutions which optimize or achieve a desired or suitable balance between the favourable properties provided by the humanizing substitutions on the one hand and the favourable properties of naturally occurring VHH domains on the other hand. Such methods are known by the skilled addressee. A human consensus sequence can be used as target sequence for humanization, but also other means are known in the art. One alternative includes a method wherein the skilled person aligns a number of human germline alleles, such as for instance but not limited to the alignment of IGHV3 alleles, to use said alignment for identification of residues suitable for humanization in the target sequence. Also a subset of human germline alleles most homologous to the target sequence may be aligned as starting point to identify suitable humanisation residues. Alternatively, the VHH is analyzed to identify its closest homologue in the human alleles, and used for humanisation construct design. A humanisation technique applied to Camelidae VHHs may also be performed by a method comprising the replacement of specific amino acids, either alone or in combination. Said replacements may be selected based on what is known from literature, are from known humanization efforts, as well as from human consensus sequences compared to the natural VHH sequences, or the human alleles most similar to the VHH sequence of interest. As can be seen from the data on the VHH entropy and VHH variability given in Tables A-5-A-8 of WO 08/020079, some amino acid residues in the framework regions are more conserved between human and Camelidae than others. Generally, although the invention in its broadest sense is not limited thereto, any substitutions, deletions or insertions are preferably made at positions that are less conserved. Also, generally, amino acid substitutions are preferred over amino acid deletions or insertions. For instance, a human-like class of Camelidae single domain antibodies contain the hydrophobic FR2 residues typically found in conventional antibodies of human origin or from other species, but compensating this loss in hydrophilicity by other substitutions at position 103 that substitutes the conserved tryptophan residue present in VH from double-chain antibodies. As such, peptides belonging to these two classes show a high amino acid sequence homology to human VH framework regions and said peptides might be administered to a human directly without expectation of an unwanted immune response therefrom, and without the burden of further humanisation. Indeed, some Camelidae VHH sequences display a high sequence homology to human VH framework regions and therefore said VHH might be administered to patients directly without expectation of an immune response therefrom, and without the additional burden of humanization.

Suitable mutations, in particular substitutions, can be introduced during humanization to generate a polypeptide with reduced binding to pre-existing antibodies (reference is made for example to WO 2012/175741 and WO2015/173325), for example at least one of the positions: 11, 13, 14, 15, 40, 41, 42, 82, 82a, 82b, 83, 84, 85, 87, 88, 89, 103, or 108. The amino acid sequences and/or VHH of the invention may be suitably humanized at any framework residue(s), such as at one or more Hallmark residues (as defined below) or at one or more other framework residues (i.e. non-Hallmark residues) or any suitable combination thereof. Depending on the host organism used to express the amino acid sequence, VHH or polypeptide of the invention, such deletions and/or substitutions may also be designed in such a way that one or more sites for posttranslational modification (such as one or more glycosylation sites) are removed, as will be within the ability of the person skilled in the art. Alternatively, substitutions or insertions may be designed so as to introduce one or more sites for attachment of functional groups (as described herein), for example to allow site-specific pegylation.

In some cases, at least one of the typical Camelidae hallmark residues with hydrophilic characteristics at position 37, 44, 45 and/or 47 is replaced (see WO2008/020079 Table A-03). Another example of humanization includes substitution of residues in FR 1, such as position 1, 5, 11, 14, 16, and/or 28; in FR3, such as positions 73, 74, 75, 76, 78, 79, 82b, 83, 84, 93 and/or 94; and in FR4, such as position 10 103, 104, 108 and/or 111 (see WO2008/020079 Tables A-05-A08; all numbering according to the Kabat).

### Oncolytic Virus

The term “oncolytic,” as used herein, refers to a tumor selective replicating virus which induces cell death in the infected cancer cell and/or tissue. Although normal or non-tumor cells may be infected, cancer cells are infected and selectively undergo cell death, in comparison to the normal or non-cancer cells of a subject.

The term “cell death,” as used herein, includes all forms of cell death, including for example cell lysis and/or apoptosis.

The oncolytic virus may be any type of oncolytic virus, for example derived from adenovirus, herpes simplex virus, measles virus, lentivirus, retrovirus, cytomegalovirus, baculovirus, adeno-associated virus, myxoma virus, vesicular stomatitis virus, poliovirus, Newcastle disease virus, parvovirus, coxsackievirus, Senecavirus, vaccinia virus, or orthopoxvirus. Preferably, the oncolytic virus is derived from vaccinia virus, herpes simplex virus, or adenovirus. It may be a native virus that is naturally oncolytic or may be engineered by modifying one or more viral genes so as to increase tumor selectivity and/or preferential replication in dividing cells, such as those involved in DNA replication, nucleic acid metabolism, host tropism, surface attachment, virulence, lysis and spread (see for example Kirn et al., 2001, Nat. Med. 7:781; Wong et al., 2010, Viruses 2:78-106). One may also envisage placing one or more viral gene(s) under the control of event or tissue-specific regulatory elements (e.g. promoter).

In one embodiment, the oncolytic virus of the present invention is obtained from a reovirus. A representative example includes Reolysin (under development by Oncolytics Biotech; NCT01166542).

In one embodiment, the oncolytic virus of the present invention is obtained from a Seneca Valley virus. A representative example includes NTX-010 (Rudin et al., 2011, Clin. Cancer. Res. 17 (4): 888-95).

In one embodiment, the oncolytic virus of the present invention is obtained from a vesicular stomatitis virus (VSV). Representative examples are described in the literature (e.g. Stojdl et al., 2000, Nat. Med. 6 (7): 821-5; Stojdl et al., 2003, Cancer Cell 4 (4): 263-75).

In one embodiment, the oncolytic virus of the present invention is obtained from a Newcastle disease virus. Representative examples include without limitation the 73-T PV701 and HDV-HUJ strains as well as those described in the literature (e.g. Phuangsab et al., 2001, Cancer Lett. 172 (1): 27-36; Lorence et al., 2007, Curr. Cancer Drug Targets 7 (2): 157-67; Freeman et al., 2006, Mol. Ther. 13 (1): 221-8).

In one embodiment, the oncolytic virus of the present invention is obtained from a herpes virus. The Herpesviridae are a large family of DNA viruses that all share a common structure and are composed of relatively large double-stranded, linear DNA genomes encoding 100-200 genes encapsided within an icosahedral capsid which is enveloped in a lipid bilayer membrane. Although the oncolytic herpes virus can be derived from different types of HSV, particularly preferred are HSV1 and HSV2, more particularly HSV1. The herpes virus may be genetically modified so as to restrict viral replication to tumors or reduce its cytotoxicity in non-dividing cells. For example, any viral gene involved in nucleic acid metabolism may be inactivated, such as thymidine kinase, ribonucleotide reductase (RR) or uracil-N-glycosylase. Another aspect involves viral mutants with defects in the function of genes encoding virulence factors such as the ICP34.5 gene. Representative examples of oncolytic herpes virus include talimogene laherparepvec.

In one embodiment, the oncolytic virus of the present invention is obtained from a morbillivirus which can be obtained from the paramyxoviridae family, with a specific preference for measles virus. Representative examples of oncolytic measles viruses include without limitation MV-Edm (McDonald et al., 2006; Breast Cancer Treat. 99(2): 177-84) and HMWMAA (Kaufmann et al., 2013, J. Invest. Dermatol. 133(4): 1034-42)

In one embodiment, the oncolytic virus of the present invention is obtained from an adenovirus. Methods are available in the art to engineer oncolytic adenoviruses. An advantageous strategy includes the replacement of viral promoters with tumor-selective promoters or modifications of the El adenoviral gene product(s) to inactivate its/their binding function with p53 or retinoblastoma (Rb) protein that are altered in tumor cells. In the natural context, the adenovirus E1B55kDa gene cooperates with another adenoviral product to inactivate p53 (p53 is frequently dysregulated in cancer cells), thus preventing apoptosis. Representative examples of oncolytic adenovirus include ONYX-015 (e.g. Khuri et al., 2000, Nat. Med 6(8): 879-85) and H101 also named Oncorine (Xia et al., 2004, Ai Zheng 23(12): 1666-70).

In one embodiment, the oncolytic virus of the present invention is a poxvirus. As used herein the term “poxvirus” refers to a virus belonging to the Poxviridae family, with a specific preference for a poxvirus belonging to the Chordopoxviridae subfamily and more preferably to the Orthopoxvirus genus. Sequences of the genome of various poxviruses, for example, the vaccinia virus, cowpox virus, Canarypox virus, Ectromelia virus, Myxoma virus genomes are available in the art and specialized databases such as Genbank (accession number NC_006998, NC_003663, NC_005309, NC_004105, NC_001132 respectively).

Desirably, the oncolytic poxvirus is an oncolytic vaccinia virus. Vaccinia viruses are members of the poxvirus family characterized by a 200 kb double-stranded DNA genome that encodes numerous viral enzymes and factors that enable the virus to replicate independently from the host cell machinery. The majority of vaccinia virus particles is intracellular (IMV for intracellular mature virion) with a single lipid envelop and remains in the cytosol of infected cells until lysis. The other infectious form is a double enveloped particle (EEV for extracellular enveloped virion) that buds out from the infected cell without lysing it. Although it can derive from any vaccinia virus strain, Elstree, Wyeth, Copenhagen and Western Reserve strains are particularly preferred. The gene nomenclature used herein is that of Copenhagen vaccinia strain. It is also used herein for the homologous genes of other poxviridae unless otherwise indicated. However, gene nomenclature may be different according to the pox strain but correspondence between Copenhagen and other vaccinia strains are generally available in the literature.

In one embodiment, the oncolytic virus of this invention further expresses at least one therapeutic gene inserted in the viral genome. A “therapeutic gene” encodes a product capable of providing a biological activity when administered appropriately to a subject, which is expected to cause a beneficial effect on the course or a symptom of the pathological condition to be treated by either potentiating anti-tumor efficacy or reinforcing the oncolytic nature of the virus. In the context of the invention, the therapeutic gene can be of mammal origin (e.g. human, murine, rabbit, etc) or not (e.g. of bacterial, yeast or viral origin).

A vast number of therapeutic genes may be envisaged in the context of the invention such as those encoding polypeptides that can compensate for defective or deficient proteins in the subject, or those that act through toxic effects to limit or remove harmful cells from the body or those that encode immunity conferring polypeptides. They may be native genes or genes obtained from the latter by mutation, deletion, substitution and/or addition of one or more nucleotides. Advantageously, the oncolytic virus of the present invention carries a therapeutic gene selected from the group consisting of genes encoding suicide gene products and immunostimulatory proteins.

The term “suicide gene” refers to a gene coding for a protein able to convert a precursor of a drug into a cytotoxic compound. Suicide genes comprise but are not limited to genes coding protein having a cytosine deaminase activity, a thymidine kinase activity, an uracil phosphoribosyl transferase activity, a purine nucleoside phosphorylase activity and a thymidylate kinase activity.

As used herein, the term “immunostimulatory protein” refers to a protein which has the ability to stimulate the immune system, in a specific or non-specific way. A vast number of proteins are known in the art for their ability to exert an immunostimulatory effect. Examples of suitable immunostimulatory proteins in the context of the invention include without limitation cytokines, with a specific preference for interleukins (e.g. IL-2, IL-6, IL-12, IL-15, IL-24), chemokines (e.g. CXCL10, CXCL9, CXCL11), interferons (e.g. IFNg, IFNalpha), tumor necrosis factor (TNF), colony-stimulating factors (e.g. GM-CSF, C-CSF, M-CSF . . . ), APC (for Antigen Presenting Cell)-exposed proteins (e.g. B7.1, B7.2 and the like), growth factors (Transforming Growth Factor TGF, Fibroblast Growth Factor FGF, Vascular Endothelial Growth Factors VEGF, and the like), MHC antigens of class I or II, apoptosis inducers or inhibitors (e.g. Bax, Bcl2, BclX . . . ), cytostatic agents (p21, p16, Rb . . . ), immunotoxins, antigenic polypeptides (antigenic polypeptides, epitopes, and the like) and markers (beta-galactosidase, luciferase). Preferably, the immunostimulatory protein is an interleukin or a colony-stimulating factor, with a specific preference for GM-CSF. In another preferred embodiment, the immunostimulatory protein comprises at least one of IL-12, IL-15 or IL-15 receptor alpha subunit

In a particularly preferred embodiment, the oncolytic virus is a herpes simplex virus comprising a sequence that encodes an immunostimulatory protein, in particular an immunostimulatory protein selected from the group consisting of GM-CSF, IL-12, IL-15, and IL-15 receptor alpha subunit.

### Combination

As mentioned above, the inventors have surprisingly observed a synergistic effect when oncolytic virotherapy is combined with administration of an LTBR agonist. Administration of the LTBR agonist can be performed by administration of a direct LTBR agonist or by administration of a nucleic acid that encodes an LTBR agonist. One object of the invention is thus a combination comprising an oncolytic virus and an LTBR agonist or a nucleic acid encoding an LTBR agonist. As will be understood from the disclosures made herein, the combination of the particular oncolytic viruses and the particular LTBR agonists described herein are objects of the invention. Further thereto, the combination of oncolytic viruses that are mentioned as being preferred embodiments with LTBR agonists that are mentioned as preferred embodiment, constitute preferred embodiments in relation to the combination, compositions comprising combinations and therapies relating to such combination.

In another embodiment, the combination of the present invention further comprises one or more pharmaceutically acceptable carriers or excipients of it. In one embodiment, said one or more pharmaceutically acceptable carriers or excipients of it can be present with the Treg depletor, in particular with the CCR8 binder, and/or the LTBR agonist. Thus, the combination of the invention can either comprises a first composition comprising the Treg depletor, in particular the CCR8 binder, with said one or more pharmaceutically acceptable carriers or excipients of it and the LTBR agonist; or comprises the Treg depletor, in particular the CCR8 binder, and a second composition comprising the LTBR agonist with said one or more pharmaceutically acceptable carriers or excipients of it; or comprises said first and second compositions i.e. the Treg depletor, in particular the CCR8 binder, with said one or more pharmaceutically acceptable carriers or excipients of it and the LTBR agonist with said one or more pharmaceutically acceptable carriers or excipients of it.

Combination as used herein refers to a combination of two features (oncolytic virotherapy and LTBR agonism). These features may be present in a single entity, e.g. an oncolytic virus comprising an LTBR agonizing moiety or a nucleic acid encoding an LTBR agonist.

In one particularly preferred embodiment, the LTBR agonist is comprised by the oncolytic virus. This can be done e.g. through insertion of a transgene encoding an LTBR agonist or by binding an LTBR agonist to the oncolytic virus, e.g. by providing it as a fusion protein on a viral capsid protein.

In a further particular embodiment, the oncolytic virus comprises a nucleic acid encoding the LTBR agonist of the invention. It has been found that the provision of an LTBR agonist that comprises a single domain antibody moiety is most suitable for obtaining such an embodiment of the invention. Therefore, in a particularly preferred embodiment, the present invention provides an oncolytic virus that comprises a nucleic acid encoding an LTBR agonist comprising a single domain antibody moiety. The oncolytic virus may furthermore be engineered for increased expression of the LTBR agonist in target tissue. In another embodiment, the LTBR agonist encoded by the nucleic acid comprised by the oncolytic virus is engineered to be bound to the surface of the oncolytic virus. In yet another embodiment, the LTBR agonist encoded by the nucleic acid comprised by the oncolytic virus is engineered to be secreted from the cell that is being infected by the oncolytic virus.

In another embodiment, the LTBR agonist is a direct LTBR agonist. In particular, a direct LTBR agonist comprising a single domain antibody moiety. In a further embodiment, the present invention provides a direct LTBR agonist comprising a single domain antibody moiety and an oncolytic virus as described herein.

In another particular embodiment, the LTBR agonist and the oncolytic virus are present in a separate composition.

### Composition

Another object of the invention is a composition comprising the combination of the present invention. Thus, the composition of the invention comprises an oncolytic virus and an LTBR agonist or a nucleic acid encoding an LTBR agonist.

In another embodiment, the oncolytic virus and the LTBR agonist or nucleic acid encoding the LTBR agonist are present in a separate composition. In a further embodiment, the composition comprising the LTBR agonist is designed for administration separate from the oncolytic virus.

In a yet preferred embodiment, the composition of the invention further comprises one or more pharmaceutically acceptable carriers or excipients.

### Treatment

A further object of the invention is a combination presenting the features as described herein or a composition comprising such a combination for use as a medicine.

Another object of the invention is a combination presenting the features as described herein or a composition comprising such a combination, for use in the treatment of a cancer.

Yet another object of the invention is an oncolytic virus, presenting the features as described herein for use in the treatment of a cancer, wherein the treatment further comprises the administration of an LTBR agonist presenting the features as described herein.

Preferably, the LTBR agonist is an LTBR agonistic antibody, such as an LTBR agonistic antibody comprising a single domain antibody moiety.

Still another object of the invention is an LTBR agonist presenting the features as described herein for use in the treatment of a cancer, wherein the treatment further comprises the administration of an oncolytic virus presenting the features as described herein.

In a further embodiment the invention provides a method for treating a disease in a subject comprising administering the combination presenting the features as described herein or a composition comprising such a combination. Preferably the disease is a cancer, in particular the treatment of solid tumours.

In a further embodiment the invention provides a method for treating a disease in a subject comprising the steps of:
administering the oncolytic virus as defined herein; and
administering the LTBR agonist as defined herein,
   wherein both administrations are done separately, simultaneously or sequentially.

In another particular embodiment, the invention provides a method for treating a disease in a subject undergoing oncolytic virotherapy, the method comprising administering an LTRB agonist to said subject.

Preferably the disease is a cancer, in particular the treatment of solid tumours. In a particular embodiment, the cancer is metastatic cancer.

In a preferred embodiment of the present invention, the subject of the aspects of the invention as described herein, is a mammal, preferably a cat, dog, horse, donkey, sheep, pig, goat, cow, hamster, mouse, rat, rabbit, or guinea pig, but most preferably the subject is a human. Thus in all aspects of the invention as described herein the subject is preferably a human.

As used herein, the terms “cancer”, “cancerous”, or “malignant” refer to or describe the physiological condition on mammals that is typically characterized by unregulated cell growth.

As used herein, the term “tumour” as it applies to a subject diagnosed with, or suspected of having, a cancer refers to a malignant or potentially malignant neoplasm or tissue mass of any size, and includes primary tumours and secondary neoplasms. The terms “cancer”, “malignancy”, “neoplasm”, “tumour” and “carcinoma” can also be used interchangeably herein to refer to tumours and tumour cells that exhibit an aberrant growth phenotype characterized by a significant loss of control of cell proliferation. In general, cells of interest for treatment include precancerous (e.g. benign), malignant, pre-metastatic, metastatic, and non-metastatic cells. The teachings of the present disclosure may be relevant to any and all tumours.

Examples of tumours include but are not limited to, carcinoma, lymphoma, leukemia, blastoma, and sarcoma. More particular examples of such cancers include squamous cell carcinoma, myeloma, small-cell lung cancer, non-small cell lung cancer, glioma, hepatocellular carcinoma (HCC), hodgkin's lymphoma, non-hodgkin's lymphoma, acute myeloid leukemia (AML), multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer.

In one aspect, the tumour involves a solid tumour. Examples of solid tumours are sarcomas (including cancers arising from transformed cells of mesenchymal origin in tissues such as cancellous bone, cartilage, fat, muscle, vascular, hematopoietic, or fibrous connective tissues), carcinomas (including tumours arising from epithelial cells), mesothelioma, neuroblastoma, retinoblastoma, etc. Tumours involving solid tumours include, without limitations, brain cancer, lung cancer, stomach cancer, duodenal cancer, esophagus cancer, breast cancer, colon and rectal cancer, renal cancer, bladder cancer, kidney cancer, pancreatic cancer, prostate cancer, ovarian cancer, melanoma, mouth cancer, sarcoma, eye cancer, thyroid cancer, urethral cancer, vaginal cancer, neck cancer, lymphoma, and the like.

In another particular embodiment, the tumour is selected from the group consisting of breast invasive carcinoma, colon adenocarcinoma, head and neck squamous carcinoma, stomach adenocarcinoma, lung adenocarcinoma (NSCLC), lung squamous cell carcinoma (NSCLC), kidney renal clear cell carcinoma, skin cutaneous melanoma, esophageal cancer, cervical cancer, hepatocellular carcinoma, merkel cell carcinoma, small Cell Lung Cancer (SCLC), classical Hodgkin Lymphoma (cHL), urothelial Carcinoma, Microsatellite Instability-High (MSI-H) Cancer and mismatch repair deficient (dMMR) cancer.

In a further embodiment, the tumour is selected from the group consisting of a breast cancer, uterine corpus cancer, lung cancer, stomach cancer, head and neck squamous cell carcinoma, skin cancer, colorectal cancer, and kidney cancer. In an even further embodiment, the tumour is selected from the group consisting of breast invasive carcinoma, colon adenocarcinoma, head and neck squamous carcinoma, stomach adenocarcinoma, lung adenocarcinoma (NSCLC), lung squamous cell carcinoma (NSCLC), kidney renal clear cell carcinoma, and skin cutaneous melanoma.

As used herein, the term “administration” refers to the act of giving a drug, prodrug, antibody, or other agent, or therapeutic treatment to a physiological system (e.g. a subject or in vivo, in vitro, or ex vivo cells, tissues, and organs). Exemplary routes of administration to the human body can be through the mouth (oral), skin (transdermal), oral mucosa (buccal), ear, by injection (e.g. intravenously, subcutaneously, intratumourally, intraperitoneally, etc.) and the like. The term administration of the LTBR agonist of the invention includes direct administration of the LTBR agonist as well as indirect administration by administering a nucleic acid encoding the LTBR agonist, such that the LTBR agonist is produced from the nucleic acid in the subject. Administration of of the LTBR agonist thus includes DNA and RNA therapy methods that result in in vivo production of the LTBR agonist.

Reference to “treat” or “treating” a tumour as used herein defines the achievement of at least one therapeutic effect, such as for example, reduced number of tumour cells, reduced tumour size, reduced rate to cancer cell infiltration into peripheral organs, or reduced rate of tumour metastasis or tumour growth. As used herein, the term “modulate” refers to the activity of a compound to affect (e.g. to promote or treated) an aspect of the cellular function including, but not limited to, cell growth, proliferation, invasion, angiogenesis, apoptosis, and the like.

Positive therapeutic effects in cancer can be measured in a number of ways (e.g. Weber (2009) J Nucl Med 50, 15-10S). By way of example, with respect to tumour growth inhibition, according to National Cancer Institute (NCI) standards, a T/C≤42% is the minimum level of anti-tumour activity. A T/C<10% is considered a high anti-tumour activity level, with T/C (%)=Median tumour volume of the treated/Median tumour volume of the controlx100. In some embodiments, the treatment achieved by a therapeutically effective amount is any of progression free survival (PFS), disease free survival (DFS) or overall survival (OS). PFS, also referred to as “Time to Tumour Progression” indicates the length of time during and after treatment that the cancer does not grow, and includes the amount of time patients have experienced a complete response or a partial response, as well as the amount of time patients have experienced stable disease. DFS refers to the length of time during and after treatment that the patient remains free of disease. OS refers to a prolongation in life expectancy as compared to naive or untreated individuals or patients.

Reference to “prevention” (or prophylaxis) as used herein refers to delaying or preventing the onset of the symptoms of the cancer. Prevention may be absolute (such that no disease occurs) or may be effective only in some individuals or for a limited amount of time.

In a preferred aspect of the invention the subject has an established tumour that is the subject already has a tumour e.g. that is classified as a solid tumour. As such, the invention as described herein can be used when the subject already has a tumour, such as a solid tumour. As such, the invention provides a therapeutic option that can be used to treat an existing tumour. In one aspect of the invention the subject has an existing solid tumour. The invention may be used as a prevention, or preferably as a treatment in subjects who already have a solid tumour. In one aspect the invention is not used as a preventative or prophylaxis.

In one aspect, tumour regression may be enhanced, tumour growth may be impaired or reduced, and/or survival time may be enhanced using the invention as described herein, for example compared with other cancer treatments (for example standard-of care treatments for the a given cancer).

In one aspect of the invention the method of treatment or prevention of a tumour as described herein further comprises the step of identifying a subject who has tumour, preferably identifying a subject who has a solid tumour.

The dosage regimen of a therapy described herein that is effective to treat a patient having a tumour may vary according to factors such as the disease state, age, and weight of the patient, and the ability of the therapy to elicit an anti-cancer response in the subject. Selection of an appropriate dosage will be within the capability of one skilled in the art. In certain embodiments, the LTBR agonist is administered in 0.01, 0.1, 0.3, 0.5, 1, 2, 3, 4, 5, 6, 7, 8, 9, 10, 20, 30, 40, or 50 mg/kg doses. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Unit dose of the present invention may conveniently be described in terms of plaque forming units (pfu) or viral particles for viral constructs. Unit doses range from 10³, 10⁴, 10⁵, 10⁶, 10⁷, 10⁸, 10⁹, 10¹⁰, 10¹¹, 10¹², 10¹³ pfu or vp and higher. Alternatively, depending on the kind of virus and the titer attainable, one will deliver 1 to 100, 10 to 50, 100-1000, or up to about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, or 1×10¹⁵ or higher infectious viral particles (vp) to the patient or to the patient's cells.

The combination, the composition and the bispecific molecule according to any aspect of the invention as described herein, may be in the form of a pharmaceutical composition which additionally comprises a pharmaceutically acceptable carrier, diluent or excipient. As used herein, the term “pharmaceutically acceptable carrier” or “pharmaceutically acceptable excipient” includes any material which, when combined with an active ingredient, allows the ingredient to retain biological activity.

Pharmaceutically acceptable carriers enhance or stabilize the composition or can be used to facilitate preparation of the composition. Pharmaceutically acceptable carriers include solvents, dispersion media, coatings, antibacterial and antifungal agents, isotonic and absorption delaying agents, and the like that are physiologically compatible, as is known to those skilled in the art (see, for example, Remington's Pharmaceutical Sciences, 18th Ed. Mack Printing Company, 1990, pp. 1289-1329; Remington: The Science and Practice of Pharmacy, 21st Ed. Pharmaceutical Press 2011; and subsequent versions thereof). Non-limiting examples of said pharmaceutically acceptable carrier comprise any of the standard pharmaceutical carriers such as a phosphate buffered saline solution, water, emulsions such as oil/water emulsion, and various types of wetting agents.

These compositions include, for example, liquid, semi-solid and solid dosage formulations, such as liquid solutions (e.g., injectable and infusible solutions), dispersions or suspensions, tablets, pills, or liposomes. In some embodiments, a preferred form may depend on the intended mode of administration and/or therapeutic application. Pharmaceutical compositions containing the combination or the composition can be administered by any appropriate method known in the art, including, without limitation, oral, mucosal, by-inhalation, topical, buccal, nasal, rectal, or parenteral (e.g. intravenous, infusion, intratumoural, intranodal, subcutaneous, intraperitoneal, intramuscular, intradermal, transdermal, or other kinds of administration involving physical breaching of a tissue of a subject and administration of the pharmaceutical composition through the breach in the tissue). Such a formulation may, for example, be in a form of an injectable or infusible solution that is suitable for intradermal, intratumoural or subcutaneous administration, or for intravenous infusion. In a particular embodiment, the oncolytic virus is administered by injection, particularly intratumoral injection. If the LTBR agonist or nucleic acid encoding the LTBR agonist is administered separately, in a particular embodiment, it is administered by injection, particularly by intratumoral injection. In another embodiment, the LTRB agonist or nucleic acid encoding the LTBR agonist is administered systemically, particularly by intravenous injection. The administration may involve intermittent dosing. Alternatively, administration may involve continuous dosing (e.g., perfusion) for at least a selected period of time, simultaneously or between the administration of other compounds.

Formulations of the invention generally comprise therapeutically effective amounts of the oncolytic virus and the LTBR agonist as defined in the combination of the invention. “Therapeutic levels”, “therapeutically effective amount” or “therapeutic amount” means an amount or a concentration of an active agent that has been administered that is appropriate to safely treat the condition to reduce or prevent a symptom of the condition.

In some embodiments, the LTBR agonist or nucleic acid encoding the LTBR agonist as defined in the combination of the present invention can be prepared with carriers that protect it against rapid release and/or degradation, such as a controlled release formulation, such as implants, transdermal patches, and microencapsulated delivery systems. Biodegradable, biocompatible polymers can be used.

Those skilled in the art will appreciate, for example, that route of delivery (e.g., oral vs intravenous vs subcutaneous vs intratumoural, etc) may impact dose amount and/or required dose amount may impact route of delivery. For example, where particularly high concentrations of an agent within a particular site or location (e.g., within a tumour) are of interest, focused delivery (e.g., in this example, intratumoural delivery) may be desired and/or useful. Other factors to be considered when optimizing routes and/or dosing schedule for a given therapeutic regimen may include, for example, the particular cancer being treated (e.g., type, stage, location, etc.), the clinical condition of a subject (e.g., age, overall health, etc.), the presence or absence of combination therapy, and other factors known to medical practitioners. In a particular embodiment, the oncolytic virus is administered intratumoral. In another particular embodiment, the LTBR agonist is administered intravenously. In a further particular embodiment, the oncolytic virus is administered by intratumoral injection and the LTBR agonist is administered by intratumoral injection.

The pharmaceutical compositions typically should be sterile and stable under the conditions of manufacture and storage. The composition can be formulated as a solution, microemulsion, dispersion, liposome, or other ordered structure suitable to high drug concentration. Sterile injectable solutions can be prepared by incorporating the binder in the required amount in an appropriate solvent with one or a combination of ingredients enumerated above, as required, followed by filtered sterilization. Formulations for parenteral administration include, but are not limited to, suspensions, solutions, emulsions in oily or aqueous vehicles, pastes, and implantable sustained-release or biodegradable formulations as discussed herein. Sterile injectable formulations may be prepared using a non-toxic parenterally acceptable diluent or solvent. Each pharmaceutical composition for use in accordance with the present invention may include pharmaceutically acceptable dispersing agents, wetting agents, suspending agents, isotonic agents, coatings, antibacterial and antifungal agents, carriers, excipients, salts, or stabilizers are non-toxic to the subjects at the dosages and concentrations employed. Preferably, such a composition can further comprise a pharmaceutically acceptable carrier or excipient for use in the treatment of cancer that that is compatible with a given method and/or site of administration, for instance for parenteral (e.g. subcutaneous, intradermal, or intravenous injection), intratumoural, or peritumoural administration.

While an embodiment of the treatment method or compositions for use according to the present invention may not be effective in achieving a positive therapeutic effect in every subject, it should do so in a using pharmaceutical compositions and dosing regimens that are consistently with good medical practice and statistically significant number of subjects as determined by any statistical test known in the art such as the Student's t-test, the X²-test, the U-test according to Mann and Whitney, the Kruskal-Wallis test (H-test), Jonckheere-Terpstra test and the Wilcoxon-test.

Where hereinbefore and subsequently a tumour, a tumour disease, a carcinoma or a cancer is mentioned, also metastasis in the original organ or tissue and/or in any other location are implied alternatively or in addition, whatever the location of the tumour and/or metastasis is.

In some embodiments, a different agent against cancer may be administered in combination with the combination or the composition of the invention via the same or different routes of delivery and/or according to different schedules. Alternatively or additionally, in some embodiments, one or more doses of a first active agent is administered substantially simultaneously with, and in some embodiments via a common route and/or as part of a single composition with, one or more other active agents. Those skilled in the art will further appreciate that some embodiments of combination therapies provided in accordance with the present invention achieve synergistic effects; in some such embodiments, dose of one or more agents utilized in the combination may be materially different (e.g., lower) and/or may be delivered by an alternative route, than is standard, preferred, or necessary when that agent is utilized in a different therapeutic regimen (e.g., as monotherapy and/or as part of a different combination therapy).

In some embodiments, where two or more active agents are utilized in accordance with the present invention, such agents can be administered simultaneously or sequentially. In some embodiments, administration of one agent is specifically timed relative to administration of another agent. For example, in some embodiments, a first agent is administered so that a particular effect is observed (or expected to be observed, for example based on population studies showing a correlation between a given dosing regimen and the particular effect of interest). In some embodiments, desired relative dosing regimens for agents administered in combination may be assessed or determined empirically, for example using ex vivo, in vivo and/or in vitro models; in some embodiments, such assessment or empirical determination is made in vivo, in a patient population (e.g., so that a correlation is established), or alternatively in a particular patient of interest.

“In combination” or treatments comprising administration of a further therapeutic may refer to administration of the additional therapy before, at the same time as or after administration of any aspect according to the present invention. Combination treatments can thus be administered simultaneous, separate or sequential.

In another embodiment, the invention provides a kit comprising the combination or the composition described above. In some embodiments, the kit further contains a pharmaceutically acceptable carrier or excipient of it. In other related embodiments, any of the components of the above combinations in the kit are present in a unit dose, in particular the dosages as described herein. In a yet further embodiment, the kit includes instructions for use in administering any of the components or the above combinations to a subject. In one particular embodiment, the kit comprises an oncolytic virus as described herein and an LTBR agonist. The oncolytic virus and the LTBR agonist can be present in the same or in a different composition.

In one particular embodiment, the present invention provides a package comprising a combination, a composition and/or a bispecific molecule as described herein, wherein the package further comprises a leaflet with instructions to administer the LTBR agonist or nucleic acid encoding the LTBR agonist to a tumour patient that also receives treatment with an oncolytic virus.

In yet another particular embodiment, the present invention provides the use of an LTBR agonist or nucleic acid encoding the LTBR agonist for the manufacture of a medicament for the treatment of a disease as described herein, wherein the treatment further comprises administration of an oncolytic virus as described herein. In another particular embodiment, the present invention provides the use of an oncolytic virus as described herein for the manufacture of a medicament for the treatment of a disease as described herein, wherein the treatment further comprises administration of an LTBR agonist or nucleic acid encoding the LTBR agonist. In another further embodiment, the present invention provides the use of an LTRB agonist or nucleic acid encoding the LTBR agonist and an oncolytic virus as described herein for the manufacture of a medicament for the treatment of a disease as described herein. The present invention further provides pharmaceutical compositions as described herein for the treatment of a disease as described herein, particularly cancer.

The invention will now be further described by way of the following examples, which are meant to serve to assist one of ordinary skill in the art in carrying out the invention and are not intended in any way to limit the scope of the invention, with reference to the drawings.

### EXAMPLES

The following examples are provided in order to demonstrate and further illustrate certain preferred embodiments and aspects of the present invention and are not construed as limiting the scope thereof.

### Example 1: LTBR Reporter Assay

### Generation of Stable LTBR Reporter Cell Line

A transgenic constructs was generated, carrying a mouse-human chimera LTBR coding sequence in which the intracellular part of the mouse orthologue was replaced by the human counterpart to ensure functional signaling in a human cell line background. A human NFκB Luciferase Reporter HEK293 stable cell line (Signosis, cat. #SL-0012) was cultured at 37° C. and 5% CO2 in Dulbecco's Modified Eagle Medium (DMEM, Gibco) supplemented with 10% heat-inactivated fetal bovine serum (FBS) and 100 U/mL penicillin and streptomycin (Gibco). Before transfection, cells were seeded at a density of 7.5×10⁵ cells per well of 6-well plates (Greiner) and cultured overnight. Upon reaching an approximate confluence of 40%, cells were transfected with linearized pcDNA3.1 carrying the mouse-human chimera LTBR transgene, using FUGENE HD transfection reagent (Promega). After 6 hours, cellular supernatants were carefully removed and replaced by fresh complete DMEM. After 48 hours, culture medium was replaced to include 500 μg/mL G-418 (Thermofisher Scientific) to select for geneticin-resistant transfectants harboring the expression cassette. Medium was changed every 2-3 days and after 3 weeks, limiting 1:2 dilutions were made starting from 10³ cells per well to obtain monoclonal lines. Identification of LTBR-expressing monoclonal lines was based on acquiring 10⁴ cells in flow cytometry (Attune NxT, Thermofisher Scientific) using a phycoerythrin-labelled mouse anti-mouse LTBR mAb 5G11 (Abcam, cat. #ab65089).

### Reporter Assay

Cells were plated in Poly-D-Lysine (PDL) coated 96-well plates (Greiner) at a density of 6.0×10⁴ cells/well and cultured overnight at 37° C. and 5% CO₂ in Dulbecco's Modified Eagle Medium (DMEM, Gibco) supplemented with 10% heat-inactivated fetal bovine serum (FBS) and 100 U/mL penicillin and streptomycin (Gibco). Compounds (VHHs and mAbs) were incubated at different concentrations for 6 hours to evaluate their agonistic activity on LTBR to induce NFκB transcription. Luciferase activity was measured using the Steadylite plus Reporter Gene Assay System (PerkinElmer, cat. #6066756) according to the manufacterer's instructions, on an EnSight™ Multimode Plate Reader (PerkinElmer). Final QC of the stable reporter cell line was done by means of a titration of the agonistic anti-mouse LTBR mAb 5G11 (Abcam, cat. #ab65089) which activates the reporter in a dose-dependent manner.

### Example 2: Generation of LTBR Agonistic Single Domain Antibody Moieties

### Immunizations

VHHs were generated through immunization of llamas and alpacas with recombinant protein, essentially as described elsewhere (Pardon et al., 2014) (Henry and Mackenzie, 2018). Briefly, animals were immunized six times at one week intervals with 50 μg of recombinant mouse LTBR—mouse IgG2A Fc chimera protein (R&D Systems, cat. #1008-LR) after which blood samples were taken.

### Phage Display Library Preparation

Phage display libraries derived from peripheral blood mononuclear cells (PBLCs) were prepared and used as described elsewhere (Pardon et al., 2014; Henry and Mackenzie, 2018). The VHH fragments were inserted into a M13 phagemid vector containing MYC and His6 tags. The libraries were rescued by infecting exponentially-growing Escherichia coli TG1 [(F′ traD36 proAB IaclqZ AM15) supE thi-1 Δ(Iac-proAB)Δ(mcrB-hsdSM)5(rK-mk-)] cells followed by surinfection with VCSM13 helper phage. The mouse LTBR immunized phage libraries were subjected to two consecutive selection rounds on mouse LTBR-mouse IgG2A Fc chimera protein (R&D Systems, cat. #1008-LR), in the presence of a 50-fold excess of total mouse IgG to eliminate Fc-binding VHHs. Individual colonies were grown in 96-deep-well plates from E. coli TG1 cells that were infected with the eluted phages from the different selection rounds. Monoclonal VHHs were expressed essentially as described before (Pardon et al., 2014). The crude periplasmic extracts containing the VHHs were prepared by freezing the bacterial pellets overnight followed by resuspension in PBS and centrifugation to remove cell debris.

### Screening of LTBR Selection Outputs

VHHs clones from the immunization and selection campaign were screened as crude periplasmic extracts by means of binding ELISA to mouse LTBR compared to uncoated controls. Binding was confirmed by means of biolayer interferometry

### ELISA

1 μg/ml of mLTBR-mFc (R&D Systems, cat. #1008-LR) diluted in PBS at pH 7.4 was coated on 96-well microtiter plates followed by blocking with 4% dry skimmed milk in PBS (Marvel). Next, 1:5 dilutions of crude periplasmic extracts from monoclonal VHH clones were added, followed by detection with 1:1000 anti-c-myc antibody 9E10 (Merck, cat. #11667203001) and anti-mouse IgG-HRP (Jackson Immuno Research, cat. #715-035-150) at a 1:5000 dilution, both in 1% dry skimmed milk in PBS. In between applications, plates were washed with PBS supplemented with Tween 0.05% pH7.4. Reaction development was done using 100μl of HRP substrate TMB (Thermo Fisher, cat. #00-4201-56). The reaction was stopped by addition of 100 μl 0.5 M H₂SO₄ (Fisher Scientific, cat. #J/8430/15) and read out on a plate reader at OD₄₅₀. Clone P002MP07G04 had an OD₄₅₀ binding signal of 4.458 to mLTBR-mFc versus 0.042 on the uncoated control.

### Biolayer Interferometry (BLI)

Bio-Layer Interferometry (BLI) is a label-free technology for measuring biomolecular interactions that analyzes the interference pattern of white light reflected from two surfaces, a layer of immobilized protein on the biosensor tip and an internal reference layer. Any change in the number of molecules bound to the biosensor tip causes a shift in the interference pattern that can be measured in real-time. The binding between a ligand immobilized on the biosensor tip surface and an analyte in solution produces an increase in optical thickness at the biosensor tip, which results in a wavelength shift, which is a direct measure of the change in thickness of the biological layer. Kinetic binding parameters off-rate (k_{off}) and dissociation constant (K_{D}) were determined on an Octet RED96e machine (ForteBio) according to the manufacturer's procedures and analyzed using the Data Analysis 9.0 software (ForteBio). Mouse LTBR-Fc (R&D Systems, cat. #1008-LR) captured on anti-murine IgG Fc capture (ForteBio, cat. #18-5088) tips was dipped in 1/5 diluted periplasmic extract of clone P002MP07G04, resulting in a k_{off} value of 1.8×10⁻⁰²S⁻¹.

### Reporter Assay

Clone P002MP07G04 was displayed in multimeric fashion on top of monoclonal phage particles, and screened in the reporter assay to evaluate its agonistic potential in comparison to irrelevant controls. Two different formats of monoclonal phages were thus evaluated: (i) VCSM13-rescued phages that display a range (one to five) of VHH fragments per phage particle and (ii) Hyperphage-rescued phages (Progen, cat. #PRHYPE-XS) that display five VHH fragments per phage particle. Clone P002MP07G04 thus yielded a reporter assay signal ratio compared to an irrelevant control of respectively 4.7 and 3.2, suggesting that a multivalent display of P002MP07G04 is able to activate mouse LTBR.

### Production, Purification and In Vitro Characterization of Monovalent LTBR VHHs

Synthetic DNA fragments encoding VHHs were ordered and subcloned into an E. coli expression vector under control of an IPTG-inducible lac promoter, in frame with N-terminal PelB signal peptide (which directs the recombinant proteins to the periplasmic compartment) and C-terminal FLAG3 and HIS6 tags. Electrocompetent E. coli TG1 cells were transformed and the resulting clones were sequence verified. VHH proteins were purified from these clones by means of IMAC chromatography followed by desalting according to well established procedures (Pardon et al., 2014).

A binding KD of 55 nM for purified monovalent P002MP07G04 to mouse LTBR-Fc (R&D Systems, cat. #1008-LR) captured on anti-murine IgG Fc capture (ForteBio, cat. #18-5088) tips was determined by means of BLI.

100 nM of purified monovalent P002MP07G04 was cross-linked through its C-terminal HIS6 tag by an anti-His tag mAb (Genscript, cat. #A00186-100) at a 2:1 molar ratio. This dimeric display of P002MP07G04 imparted LTBR agonism in the reporter assay with an NFκB signal to background ratio of 6.8. In contrast, non-cross-linked monovalent P002MP07G04 was not active at 100 nM in the reporter assay.

### Production, Purification and in Vitro Characterization of Multivalent LTBR VHHs

VHH-16, a tetravalent VHH combining three P002MP07G04 building blocks and one anti-serum albumin building block SA26h5 (WO/2019/016237), separated by 20GS flexible GlySer linkers, was generated essentially as described before (Maussang et al., 2013; De Tavernier et al., 2016). The multivalent construct was cloned and sequence-verified in a Pichia pastoris expression vector under control of an AOX1 methanol-inducible promoter, in frame with an N-terminal Saccharomyces cerevisiae alpha mating factor signal peptide that directs the expressed recombinant proteins to the extracellular environment. Transformation and expression in Pichia pastoris and purification by means of protein A purification were done essentially as described before (Lin-Cereghino et al., 2005; Schotte et al., 2016). When tested in the reporter assay, VHH-16 activated mouse LTBR with a mean (±standard deviation) pEC50 value of 9.35±0.03 (n=3).

1. A Lymphtoxin Beta Receptor (LTBR) agonist or a nucleic acid encoding an LTBR agonist for use in the treatment of a cancer, wherein the treatment comprises administration of an oncolytic virus.

2. The nucleic acid encoding an LTBR agonist for the use according to claim 1, wherein the nucleic acid encoding the LTBR agonist is comprised by the oncolytic virus.

3. The LTBR agonist or the nucleic acid encoding an LTBR agonist for the use according to claim 1, wherein the LTBR agonist or the nucleic acid encoding an LTBR agonist is separate from the oncolytic virus.

4. The LTBR agonist or the nucleic acid encoding an LTBR agonist for the use according to any one of the previous claims, wherein the LTBR agonist comprises a proteinaceous moiety that binds to LTBR.

5. The LTBR agonist or the nucleic acid encoding an LTBR agonist for the use according to any one of the previous claims, wherein the LTBR agonist comprises an antibody or fragment thereof that binds to LTBR.

6. The LTBR agonist or the nucleic acid encoding an LTBR agonist for the use according to any one of the previous claims, wherein the LTBR agonist comprises a single domain antibody moiety that binds to LTBR.

7. The LTBR agonist or the nucleic acid encoding an LTBR agonist for the use according to claim 3, wherein the LTBR agonist or the nucleic acid encoding an LTBR agonist is administered separately from the oncolytic virus.

8. The LTBR agonist or the nucleic acid encoding an LTBR agonist for the use according to any one of the previous claims, wherein the treatment further comprises administration of an anti-angiogenic agent or an immune checkpoint inhibitor.

9. An oncolytic virus comprising a nucleic acid sequence encoding an LTBR agonist.

10. The oncolytic virus according to claim 9, wherein the LTBR agonist comprises a single domain antibody moiety that binds to LTBR.

11. The oncolytic virus according to claim 9 or 10, wherein the oncolytic virus is a modified herpes simplex virus or adenovirus.

12. A combination of (a) an LTBR agonist or a nucleic acid encoding an LTBR agonist, and (b) an oncolytic virus.

13. A pharmaceutical composition comprising the oncolytic virus according to any one of claims 9 to 11, or the combination according to claim 12.

14. The pharmaceutical composition according to claim 13, for use in the treatment of a cancer.

15. A method for treating cancer in a subject in need thereof, the method comprising administering an LTBR agonist or a nucleic acid encoding an LTBR agonist, and an oncolytic virus to said subject.
